(19) United States
(12) Patent Application Publication
(10) Pub. No.: US 2024/0325600 A1
(43) Pub. Date: Oct. 3, 2024

(54) POROUS BIOLOGICAL AMNIOTIC MEMBRANE PUNCTURE DEVICE AND PREPARATION METHOD

(71) Applicant: Chinese PLA General Hospital, Beijing (CN)

(72) Inventors: Chunbao LI, Beijing (CN); Jiating ZHANG, Beijing (CN); Yujie LIU, Beijing (CN); Wei CHEN, Beijing (CN); Ying SUN, Beijing (CN)

(73) Assignee: Chinese PLA General Hospital, Beijing (CN)

(21) Appl. No.: 18/621,390

(22) Filed: Mar. 29, 2024

(30) Foreign Application Priority Data
Mar. 30, 2023 (CN) 202310324768.3

(51) Int. Cl. A61L 27/36; A61F 2/30; A61L 27/56

(52) U.S. Cl. CPC A61L 27/3604; A61F 2/3094; A61L 27/365; A61L 27/3691; A61L 27/56; A61F 2002/3097

(57) ABSTRACT

The present disclosure belongs to the technical field of amniotic membrane, and in particular, relates to a porous biological amniotic membrane puncture device and a preparation method. The device includes a sliding table, a light-shielding frame, a base I, and a base II. The sliding table is provided with a first sliding groove; the light-shielding frame is slidably connected to the first sliding groove; a three-axis movable platform is disposed on the base I; the three-axis movable platform is fixedly connected to a laser puncture device; an upper end of the base II is fixedly connected to a fixed board surface; a middle portion of the fixed board surface is provided with an embedded groove; and an amniotic membrane sample is placed in the embedded groove.

### CROSS-REFERENCE TO RELATED APPLICATION

This application claims priority to Chinese Patent Application No. 202310324768.3, filed Mar. 30, 2023, which is incorporated herein by reference in its entirety.

### TECHNICAL FIELD

The present disclosure belongs to the technical field of amniotic membrane, and in particular, relates to a porous biological amniotic membrane puncture device and a preparation method.

### BACKGROUND

Bone injuries due to various causes have a serious impact on people's normal work and lives, and the effect of bone repair is severely restricted by the fact that the autogenous bone is prone to secondary trauma, and side effects such as nerve damage or infection.

### Amniotic Membrane has the Following Characteristics:

Biocompatibility: it may be directly chemically bonded with the bone without preventing the normal activity of osteocyte on its surface or interfering with the natural regenerative process of the surrounding osteocytes, and it is conductive to decomposition and absorption of bone tissue.
Mechanical tolerance: using the trabecular bone as an example, the compressive strength shall be greater than 5 MPa, and the compression modulus is between 45-100 MPa.
Biodegradability: when it is replaced by the host bone within a certain period of time, it does not affect the repair of the bone tissue, and it has no toxic side effects.
Inducibility and regenerative properties: bone growth is stimulated or induced by the amniotic membrane itself or by adding osteoinductive factors.
Operability: it has a shape that matches the repair site, which facilitates surgical transplantation.

Existing amniotic membrane perforation technologies rely on laser beams to perform a perforation operation on the surface of the amniotic membrane. A specific sample perforation process includes: controlling, by a controller, one dimension of a two-dimensional stepping motor to move in a horizontal direction (X axis), and the other dimension to move in a vertical direction (Y axis). The sample is fixed between two identical sample holders, and the sample holders are fixed on the Y axis of the stepping motor. Procedure setting: a step length of 1 mm is set for both the X and Y axes, and a program is started by positioning a laser on a first column at the very edge of the sample. The Y axis moves at a speed of 1 mm per step with a dwell of 0.9 s. After completing the first column with the laser, the X axis moves inward by one step, and then the Y axis moves along the Y direction with the same step length and speed as mentioned above. The two-dimensional stepping motor runs repeatedly in the above manner until the perforation of the entire sample is completed. However, this method has a relatively large actual error and is affected by the change in overall tension of the amniotic membrane, resulting in rough hole edges and low perforation efficiency.

Therefore, a porous biological amniotic membrane puncture device and a preparation method are proposed to solve the above problems.

### SUMMARY

In view of the above problems, the present disclosure is intended to provide a porous biological amniotic membrane puncture device and a preparation method, which may maintain high stability and integrity.

In order to achieve the above objective, the present disclosure adopts the following technical solutions. An amniotic membrane puncture preparation device includes a sliding table, a light-shielding frame, a base I, and a base II. The sliding table is provided with a first sliding groove; the light-shielding frame is slidably connected to the first sliding groove; a three-axis movable platform is disposed on the base I; the three-axis movable platform is fixedly connected to a laser puncture device; an upper end of the base II is fixedly connected to a fixed board surface; a middle portion of the fixed board surface is provided with an embedded groove; and an amniotic membrane sample is placed in the embedded groove.

In the porous biological amniotic membrane puncture device, the laser puncture device includes a housing and a plurality of laser groups, the plurality of laser groups are integrated within the housing and arranged in parallel with each other.

In the porous biological amniotic membrane puncture device, the laser group includes a laser, an energy attenuator, a reflector, and a focusing lens, and the laser, the energy attenuator, the reflector, and the focusing lens are connected in sequence.

In the porous biological amniotic membrane puncture device, the three-axis movable platform includes an L device plate, a first movable platform, and a second movable platform; a sidewall of the three-axis movable platform is horizontally provided with a second sliding groove; the first movable platform is slidably connected to the second sliding groove; a sidewall of the first movable platform is vertically provided with a third sliding groove; the second movable platform is slidably connected to the third sliding groove; an upper end of the second movable platform is provided with a fourth sliding groove; the laser puncture device is slidably connected to the fourth sliding groove; the L device plate, the first movable platform, and the second movable platform are each provided with a drive motor set; and the drive motor sets on the L device plate, the first movable platform, and the second movable platform are configured to control the first movable platform, the second movable platform, and the laser puncture device, respectively.

In the porous biological amniotic membrane puncture device, an upper end of the laser puncture device is fixedly connected to a laser range finder, the light-shielding frame is in a shape of an inverted U; and a signal receiving board is fixedly connected on an inner top surface of the light-shielding frame.

In the porous biological amniotic membrane puncture device, two clamp plates are disposed in the embedded groove, the amniotic membrane sample is disposed between the two clamp plates, and the two clamp plates are fixed in the embedded groove through bolts.

An amniotic membrane puncture preparation method includes the following steps.

At S1, an amniotic membrane sample is fixed in an embedded groove through two clamp plates.

At S2, based on S1, a light-shielding frame is moved, and an initial position of a laser puncture device is determined through an interaction between a laser range finder and a signal receiving board.

At S3, a laser is emitted by the laser range finder under the action of a three-axis movable platform, to perform a puncture operation on the fixed amniotic membrane sample.

At S4, the position of the laser range finder is adjusted by cooperating with mutual activities of a first movable platform and a second movable platform, such that the puncture operation on the amniotic membrane sample is performed in sequence according to a set pattern.

In the amniotic membrane puncture preparation method, the three-axis movable platform operates according to signal transmission between the laser range finder and the signal receiving board.

In the amniotic membrane puncture preparation method, the light-shielding frame is moved to completely cover the laser range finder and the amniotic membrane sample.

In the amniotic membrane puncture preparation method, a puncture mode in S4 comprises synchronous irradiation by a plurality of laser groups or asynchronous irradiation by the plurality of laser groups.

Compared with the related art, the porous biological amniotic membrane puncture device and the preparation method have the following advantages.
1. In the present disclosure, through the cooperation of the disposed L device plate, first movable platform, second movable platform, and laser puncture device, an effect of controlling the laser puncture device to perform the perforation operation on an amniotic membrane is achieved using the L device plate, the first movable platform, and the second movable platform. This allows the amniotic membrane to always maintain stable without the need of active movement after being fixed. Additionally, because a plurality of emitter are disposed on the laser puncture device, perforation efficiency is effectively improved during operation, resulting in an efficient and stable effect.
2. In the present disclosure, through the cooperation of the disposed plurality of focusing lenses and three-axis movable platform, operations may be performed according to a multi-position interval batch processing method when the plurality of focusing lenses emit laser beams by using the three-axis movable platform. This eliminates the need for sequential movement on X/Y axis according to traditional methods during the perforation operation, thereby reducing the requirements for high-frequency and accurate movements, and achieving an effect of maintaining high position accuracy even under batch operations.

### BRIEF DESCRIPTION OF THE DRAWINGS

FIG. 1 is a schematic diagram of an external structure of an amniotic membrane puncture device according to the present disclosure.

FIG. 2 is a schematic structural diagram of a three-axis movable platform in FIG. 1.

FIG. 3 is a schematic structural diagram of a three-axis movable platform in FIG. 1 in another view.

FIG. 4 is a schematic diagram of a bottom structure of a light-shielding frame in FIG. 1.

FIG. 5 is a schematic structural diagram of a laser puncture device in FIG. 2.

FIG. 6 is a schematic diagram of a mounting position of a focusing lens in FIG. 5.

FIG. 7 is a schematic diagram of a fixation method of an amniotic membrane sample in FIG. 3.

In the drawings, 1 Sliding table, 2 Light-shielding frame, 3 Base I, 4 Base II, 5 First sliding groove, 6 Three-axis movable platform, 7 Laser puncture device, 8 Fixed board surface, 9 Amniotic membrane sample, 10 Focusing lens, 11L device plate, 12 First movable platform, 13 Second movable platform, 14 Second sliding groove, 15 Third sliding groove, 16 Fourth sliding groove, 17 Laser range finder, 18 Signal receiving board, 19 Clamp plate.

### DETAILED DESCRIPTION OF THE EMBODIMENTS

The following embodiments are for illustrative purposes only and are not intended to limit the scope of the present disclosure.

### EMBODIMENT

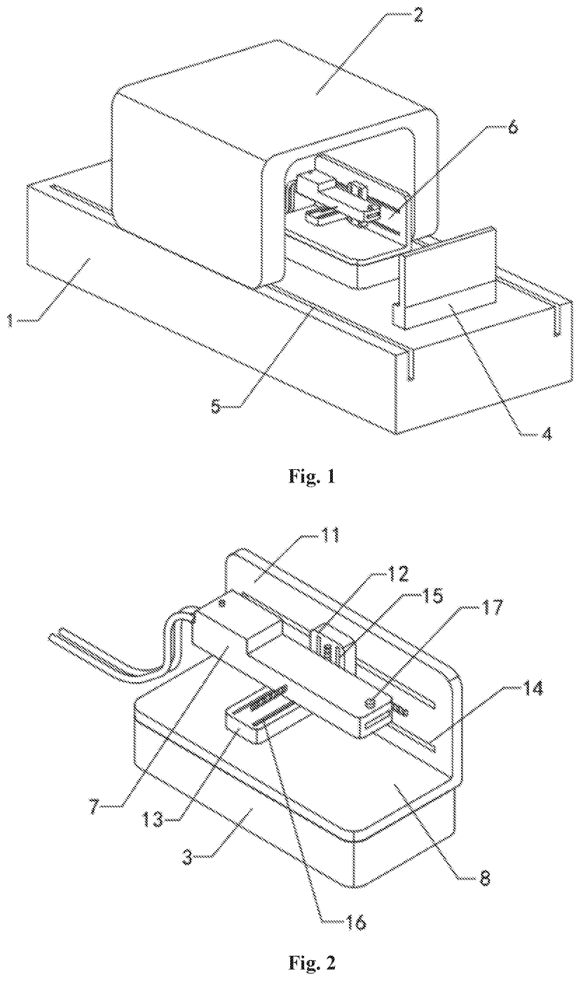

Existing amniotic membrane perforation technologies rely on laser beams to perform a perforation operation on the surface of the amniotic membrane. A specific sample perforation process includes: controlling, by a controller, one dimension of a two-dimensional stepping motor to move in a horizontal direction (X axis), and the other dimension to move in a vertical direction (Y axis). The sample is fixed between two identical sample holders, and the sample holders are fixed on the Y axis of the stepping motor. Procedure setting: a step length of 1 mm is set for both the X and Y axes, and a program is started by positioning a laser on a first column at the very edge of the sample. The Y axis moves at a speed of 1 mm per step with a dwell of 0.9 s. After completing the first column with the laser, the X axis moves inward by one step, and then the Y axis moves along the Y direction with the same step length and speed as mentioned above. The two-dimensional stepping motor runs repeatedly in the above manner until the perforation of the entire sample is completed. However, this method has a relatively large actual error and is affected by the change in overall tension of the amniotic membrane, resulting in rough hole edges and low perforation efficiency. Therefore, as shown in FIG. 1, a porous biological amniotic membrane puncture device is designed in the present solution. A main body includes a sliding table 1, a light-shielding frame 2, a base I 3, and a base II 4. The sliding table 1 is configured to support a mounting base; the light-shielding frame 2 is of a door frame structure, which runs through from front to back; the base I 3 is configured to mount a laser perforation device; and the base II 4 is configured to mount an amniotic membrane sample.

As shown in FIG. 1, the sliding table 1 is provided with a plurality of first sliding grooves 5, the first sliding grooves 5 are disposed on edges of two ends of the sliding table 1, and the light-shielding frame 2 is slidably connected to the first sliding grooves 5. As shown in FIG. 4, a self-propelled structure is mounted at the bottom of the light-shielding frame 2, and the bottom of the light-shielding frame 2 is fitted to the first sliding grooves 5, such that the light-shielding frame 2 can be controlled to move back and forth on the sliding table 1 under the action of the self-propelled structure.

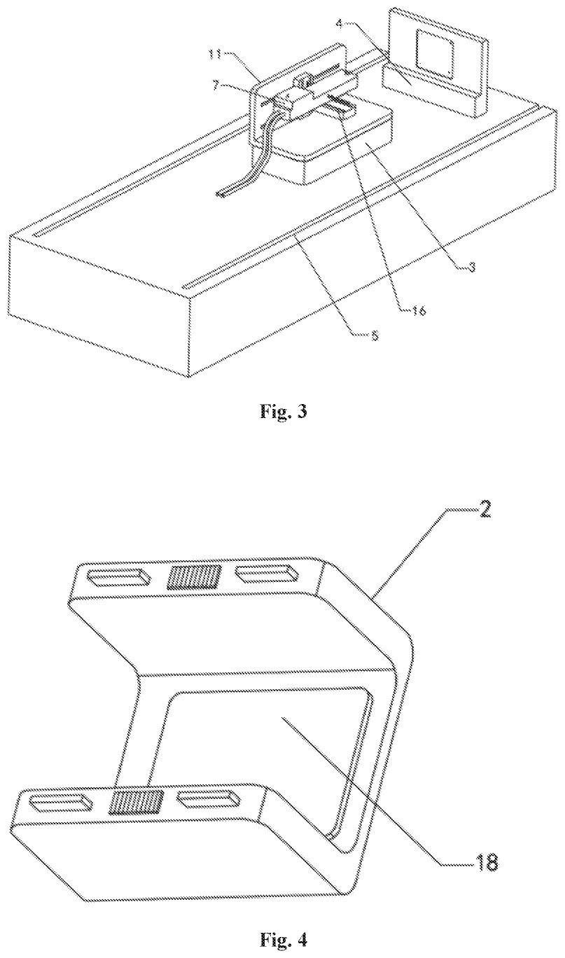

As shown in FIG. 2 to FIG. 3, a three-axis movable platform 6 is disposed on the base I 3, the three-axis movable platform 6 is configured to perform three-dimensional movement control of xyz axes, a laser puncture device 7 is fixedly connected on the three-axis movable platform 6, and the laser puncture device 7 performs autonomous multi-dimensional movement under the control of the three-axis movable platform 6. Specifically, the three-axis movable platform 6 includes an L device plate 11, a first movable platform 12, and a second movable platform 13. The L device plate 11 acts as a main support body of the entire device, and in order to achieve a support and effective use space, a sidewall of the three-axis movable platform 6 is horizontally provided with a plurality of second sliding grooves 14, and the second sliding grooves 14 and the laser puncture device 7 are parallel to each other. Furthermore, the first movable platform 12 is slidably connected to the second sliding groove 14, such that the first movable platform 12 may be moved in forward and backward positions through the second sliding groove 14. A sidewall of the first movable platform 12 is vertically provided with a third sliding groove 15, the third sliding groove 15 is perpendicular to the ground, and the second movable platform 13 is slidably connected to the third sliding groove 15, such that the second movable platform 13 may move up and down. Correspondingly, an upper end of the second movable platform 13 is provided with a fourth sliding groove 16, and the laser puncture device 7 is slidably connected to the fourth sliding groove 16, such that the laser puncture device 7 may be moved in left and right positions on the second movable platform 13.

In order to realize movement of the first movable platform 12, the second movable platform 13, and the laser puncture device 7, the L device plate 11, the first movable platform 12, and the second movable platform 13 are each provided with a structure of a drive motor set for driving, and the drive motor sets on the L device plate 11, the first movable platform 12, and the second movable platform 13 are configured to control the first movable platform 12, the second movable platform 13, and the laser puncture device 7, respectively. The specific structure of the drive motor set includes an independent drive motor and a threaded rod. The drive motor is configured to drive the threaded rod to rotate, and the threaded rod is in threaded connection with a target object (the first movable platform 12, the second movable platform 13, and the laser puncture device 7), such that under the cooperation of the second sliding groove 14, the third sliding groove 15, and the fourth sliding groove 16, a stable multi-dimensional movement effect in the xyz-axes may be achieved through the rotation of a plurality of threaded rods, so as to drive the laser puncture device 7 to achieve an effect of adjusting a plurality of positions in a single plane. Through the use of a stable self-locking structure, positional movement of the laser puncture device 7 may be accurately controlled by controlling the process of each revolution that the threaded rod rotates. The control method of the present solution adopts an external program electronic control system, which allows direct setting operations by external personnel through program control.

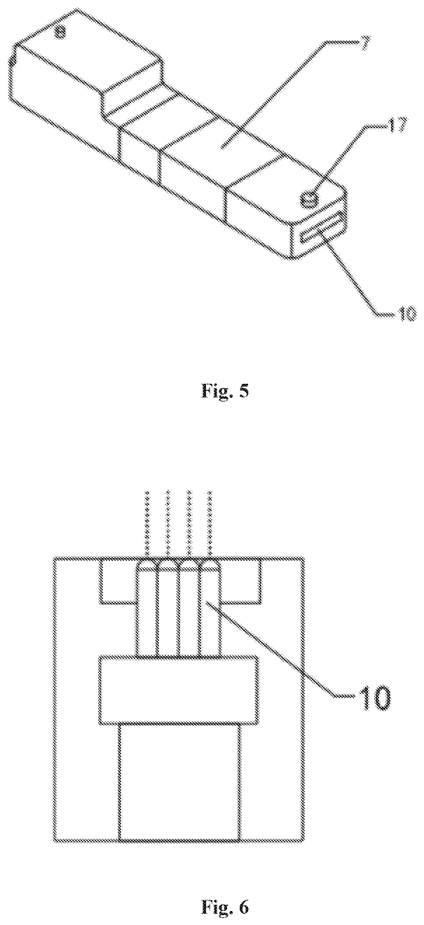

As shown in FIG. 5 to FIG. 6, the laser puncture device 7 is configured to perform perforation on an amniotic membrane. A specific structure is as follows: the laser puncture device 7 includes a housing and a laser group; the housing is configured to perform a multi-module splicing operation; a plurality of laser groups are integrated within the housing to form an integrity; the plurality of laser groups are arranged in parallel with each other to form a plurality of groups of independently-disposed unit; and the laser group includes a laser, an energy attenuator, a reflector, and a focusing lens 10, and the laser, the energy attenuator, the reflector, and the focusing lens 10 are connected in sequence. A laser emitted by the laser is attenuated to a set energy value through a plurality of reflectors and the energy attenuator, and is then emitted by the focusing lens 10. The laser puncture device 7 in the present solution is provided with 4 groups of emission heads, the 4 groups of emission heads may emit lasers independently, and spacings between the 4 groups of emission heads are constant and may be spacings (customized) of general amniotic membrane puncture. The 4 groups are not a unique option, and other numbers of groups may also be set to improve puncture efficiency.

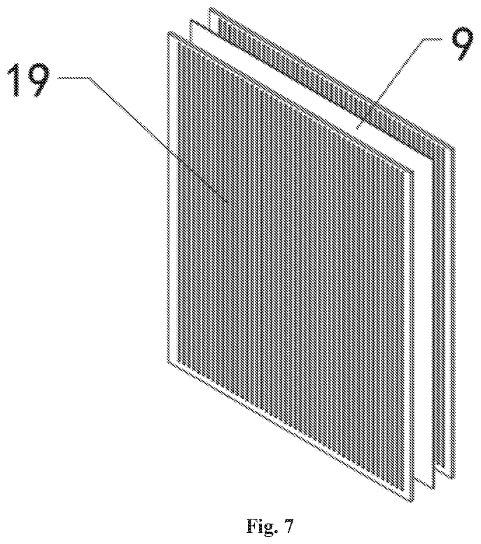

As shown in FIG. 3 to FIG. 4, an upper end of the base II 4 is fixedly connected to a fixed board surface 8, to act as a support to hold the amniotic membrane upright. An embedded groove is then provided in a middle portion of the fixed board surface 8, and an amniotic membrane sample 9 is placed in the embedded groove. The fixation method is as follows: two clamp plates 19 are disposed in the embedded groove, the amniotic membrane sample 9 is disposed between the two clamp plates 19, the two clamp plates 19 are fixed in the embedded groove through bolts, and the two clamp plates 19 are also fixed with each other through bolts. In order to solve a problem of the tension of the amniotic membrane destroying an aperture during puncture, a soft membrane is fixedly connected on an end face of each of the clamp plates 19, and the soft membranes tightly clamp the amniotic membrane sample, thereby protecting the amniotic membrane sample as much as possible from overall tension while achieving a good fixation effect. There are various sizes of the clamp plate 19, and the sizes are matched correspondingly according to different requirements. As shown in FIG. 7, the plate-like clamp plate 19 is provided with a plurality of long through stripes, and the laser puncture device 7 acts on the through stripes, so as to irradiate the surface of the amniotic membrane sample. To improve the accuracy of movement of the laser puncture device 7, a laser range finder 17 is fixedly connected to an upper end of the laser puncture device 7. The light-shielding frame 2 is in a door frame shape, and a signal receiving board 18 is fixedly connected on an inner top surface of the light-shielding frame 2. A large number of signal receivers are provided within the signal receiving board 18. The density of the signal receiving board 18 depends on moving accuracy of the laser puncture device 7 that needs to be controlled, and the density generally exceeds a movement requirement of the laser puncture device 7. The laser range finder 17 provides signal feedback through the signal receiving board 18 during positional movement, so as to determine the current position and a moving spacing. Furthermore, the laser emitted by the laser range finder 17 itself may achieve a range finding function on a vertical target, such that a three-dimensional accurate positional movement correction function can be realized.

An amniotic membrane puncture preparation method includes the following steps.

At S1, an amniotic membrane sample 9 is fixed in an embedded groove through two clamp plates 19.

At S2, based on S1, a light-shielding frame 2 is moved to completely cover a laser range finder 17 and the amniotic membrane sample 9, and an initial position of a laser puncture device 7 is determined through an interaction between the laser range finder 17 and a signal receiving board 18. The light-shielding frame 2 is configured to position and exclude interferences, such that the laser puncture device 7 can function in a stable manner. The laser puncture device 7 needs to be set with specifications according to a program and a target before functioning, such that it is operated autonomously through a control program.

At S3, a laser is emitted by the laser range finder 17 under the action of a three-axis movable platform 6, to perform a puncture operation on the fixed amniotic membrane sample 9. The three-axis movable platform 6 operates according to signal transmission between the laser range finder 17 and the signal receiving board 18, thereby achieving real-time monitoring and correction adjustment effects.

At S4, the position of the laser range finder 17 is adjusted by cooperating with mutual activities of a first movable platform 12 and a second movable platform 13, such that the puncture operation on the amniotic membrane sample 9 is performed in sequence according to a set pattern. The puncture mode includes synchronous irradiation by a plurality of laser groups or asynchronous irradiation by the plurality of laser groups. The irradiation path is generally to first irradiate an edge on one end of an amniotic membrane, then adjust a vertical spacing to puncture in sequence, and move to an edge of the other end for puncture again after the puncture ends, so as to reduce errors caused by each small movement and reduce the difficulty of accuracy control through significant movement, and then move to a set position on the other side, until all puncture operations are completed. When the irradiation of all beams of the laser puncture device 7 is not met, part of irradiation channels of the laser puncture device 7 can be autonomously closed, such that the laser puncture device emits a small number of lasers, thereby achieving an effect of the normal puncture operation.

Although terms such as the sliding table 1, the light-shielding frame 2, the base I 3, the base II 4, the first sliding groove 5, the three-axis movable platform 6, the laser puncture device 7, the fixed board surface 8, the amniotic membrane sample 9, the focusing lens 10, the L device plate 11, the first movable platform 12, the second movable platform 13, the second sliding groove 14, the third sliding groove 15, the fourth sliding groove 16, the laser range finder 17, the signal receiving board 18, and the clamp plate 19 are used herein, it does not exclude the possibility of using other terms. These terms are used only to describe and explain the essence of the present disclosure more conveniently; and to interpret the terms as any kind of additional limitation would be contrary to the spirit of the present disclosure.

1. An amniotic membrane puncture preparation method, applicable to the porous biological amniotic membrane puncture device, comprising the following steps:
S1: fixing an amniotic membrane sample (9) in an embedded groove through two clamp plates (19);
S2: based on S1, moving a light-shielding frame (2), and determining an initial position of a laser puncture device (7) through an interaction between a laser range finder (17) and a signal receiving board (18);
S3: emitting a laser by the laser range finder (17) under the action of a three-axis movable platform (6), to perform a puncture operation on the fixed amniotic membrane sample (9); and
S4: adjusting the position of the laser range finder (17) by cooperating with mutual activities of a first movable platform (12) and a second movable platform (13), such that the puncture operation on the amniotic membrane sample (9) is performed in sequence according to a set pattern.

2. The amniotic membrane puncture preparation method according to claim 1, wherein the three-axis movable platform (6) operates according to signal transmission between the laser range finder (17) and the signal receiving board (18).

3. The amniotic membrane puncture preparation method according to claim 2, wherein the light-shielding frame (2) is moved to completely cover the laser range finder (17) and the amniotic membrane sample (9).

4. The amniotic membrane puncture preparation method according to claim 3, wherein a puncture mode in S4 comprises synchronous irradiation by a plurality of laser groups or asynchronous irradiation by the plurality of laser groups.
